(19) United States
(12) Patent Application Publication
(10) Pub. No.: US 2020/0000236 A1
(43) Pub. Date: Jan. 2, 2020

(54) Neck Support Article

(71) Applicants: Frank Wu, Wayne, NJ (US); John Vantuno, Little Falls, NJ (US)

(72) Inventors: Frank Wu, Wayne, NJ (US); John Vantuno, Little Falls, NJ (US)

(21) Appl. No.: 16/351,900

(22) Filed: Mar. 13, 2019

Related U.S. Application Data
(60) Provisional application No. 62/690,459, filed on Jun. 27, 2018.

(51) Int. Cl. A47C 7/38; A47C 16/00; A61F 7/08

(52) U.S. Cl. CPC A47C 7/383; A47G 9/1081; A61F 7/08; A47C 16/00

(57) ABSTRACT

An article for supporting an individual's neck. The article is comprised of a sleeve and a support member. The support member is removably disposed within the sleeve. The support member may include a spine that is semi-ridged, and flexible ribs that are attached to the spine, such that the flexible ribs are able to support the individual's head when the article is worn. The article may also include a heating element disposed within the sleeve, configured to warm the individual's neck during use.

### CROSS REFERENCE TO RELATED APPLICATIONS

This application claims the benefit of U.S. Provisional Application No. 62/690,459 filed on Jun. 27, 2018. The above identified patent application is herein incorporated by reference in its entirety to provide continuity of disclosure

### BACKGROUND OF THE INVENTION

The present invention relates to a neck support article. More particularly, the present invention provides a neck support article comprising a sleeve and a support member, wherein the support member may be disposed within the sleeve. The support member may include a spine that is semi-ridged, and ribs that are flexibly attached to the spine, such that the ribs are able to support an individual's head when the article is worn.

Many individuals frequently travel for work or leisure. Many times, these trips mean sitting in a vehicle, train, or plane for an extended period. During, such trips individuals may grow tired and sleep in an upright or partially reclined position. Unfortunately, in such positions, an individual's head may be forced in a forward position or to the side. When kept in this position for an extended period the individual's neck and shoulder muscles may become strained and sore. This straining can cause and individual to become sore and stiff. Furthermore, many vehicles, trains, and planes can get very cold, such that an individual may seek warmth. Accordingly, a device that is configured to provide support and warmth to an individual's head, neck, and shoulders while the individual is resting in an upright position is desired.

Consequently, there is a need for an improvement in the art of neck cushions and supports. The present invention substantially diverges in design elements from the known art while at the same time solves a problem many travelers face. In this regard, the present invention substantially fulfills these needs.

### SUMMARY OF THE INVENTION

The present invention provides for a neck support article, wherein the same can be utilized for providing convenience for the user when traveling. The article includes a sleeve, wherein a middle of the sleeve is configured to removably receive a support member therein. The support member comprises a semi-rigid spine and a plurality of ribs attached to the semi-rigid spine, and the plurality of ribs is configured to support and conform to the individual's neck.

Other objects, features, and advantages of the present invention will become apparent from the following detailed description, taken in conjunction with the accompanying drawings.

### BRIEF DESCRIPTION OF THE DRAWINGS

Although the characteristic features of the invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment article with hook and loop fasteners.

FIG. 2 shows an elevated side view of an embodiment of the article with an embodiment of a support member placed therein.

FIG. 3 shows a perspective view of an embodiment of the article during use.

FIG. 4 shows a perspective view of an embodiment of the support member with a heating element attached.

FIG. 5 shows front view of an embodiment of the support member, wherein a plurality of ribs of the support member taper along a length thereof.

FIG. 6 shows perspective view an embodiment of the support member, wherein the support member includes grooves for easier bending of the support member during use.

FIG. 7 shows an embodiment of the support member, wherein the plurality of ribs is thinner, and wherein a width of the support member tapers toward the ends.

FIG. 8 shows an underside of an embodiment of the support member.

FIG. 9 shows a perspective view of an embodiment of the support member with a cross section of a single rib.

FIG. 10 shows a front view of an embodiment of the support member with many grooves running through the ribs to provide flexibility.

### DETAILED DESCRIPTION OF THE INVENTION

Reference is made herein to the attached drawings. Like reference numerals are used throughout the drawings to depict like or similar elements of the neck cushion and support article. For the purpose of presenting a brief and clear description of the present invention, an exemplary embodiment will be discussed. The figures are intended for representative purposes only and should not be considered to be limiting in any respect.

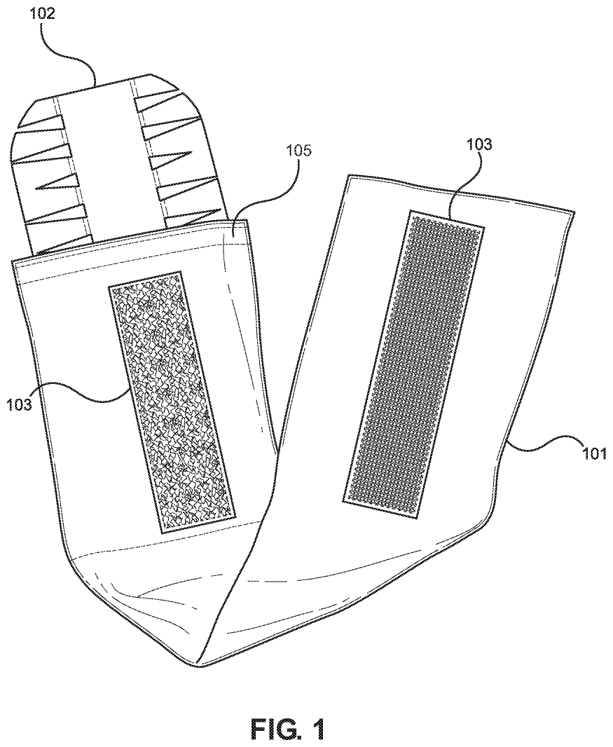

Referring now to FIG. 1, there is shown a perspective view of an embodiment article with hook and loop fasteners. The neck cushion article comprises a sleeve 101, and a support member 102. The support member 102 may be removably inserted into the sleeve 101. In the illustrated embodiment, the sleeve 101 has an opening at one end. This opening is dimensioned to receive and hold the support member 102. There is a channel defined within the sleeve 101 that is dimensioned to receive the support member 102 therein, such that the support member 102 is removably disposed within the channel. In this manner, the support member 102 may be easily removed for maintenance or cleaning of the sleeve 101.

The sleeve 101 may comprise various materials or textiles. In one embodiment cotton can be used to create the sleeve 101 be comfortable when worn. In other embodiments, the sleeve 101 comprises flannel, and in another embodiment, the sleeve 101 comprises wool for improved insulation.

The sleeve 101 includes a fastener 103 attached to two portions of the sleeve 101. The fastener 103 is used to secure the cushion around the individual's neck. In one embodiment, the fastener 103 is positioned before an end of the sleeve 101. In this manner, the sleeve 101 may be adjustable to fit around a neck having any of a range of sizes or circumferences. The fastener 103 may be, for example but not limited to, a hook and loop fastener, a button fastener, a snap fastener or a clasp fastener. In a particular embodiment, a hook and loop fastener is used in order to allow a user to adjust a size of the sleeve 101. In other embodiments, where for example snaps are used, the neck cushion can be made in different sizes or have multiple snaps placed thereon in order to allow for a more custom fit.

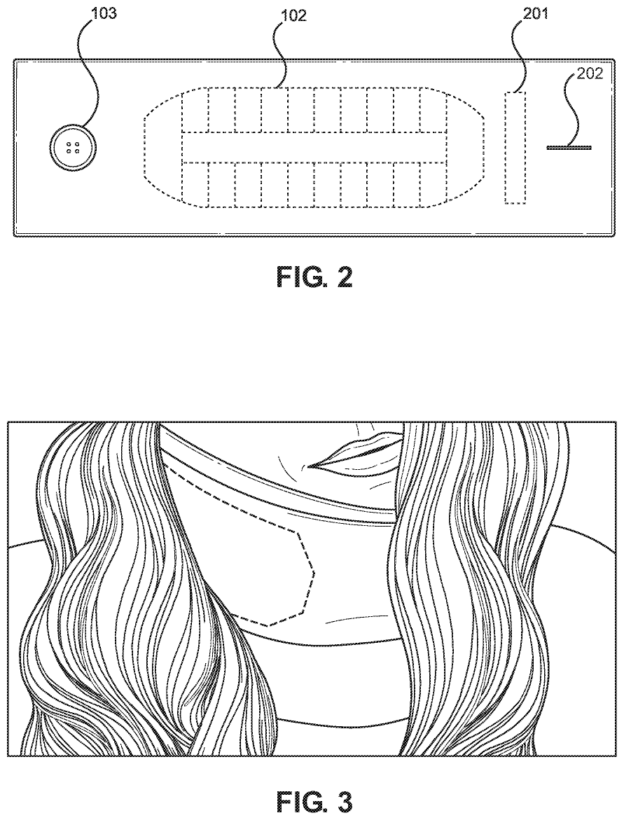

Referring now to FIG. 2, there is shown an elevated side view of an embodiment of the article with an embodiment of a support member placed therein. In the illustrated embodiment, a button fastener 103 is disposed on one end of the sleeve and a plurality of button holes 202 is disposed on a second end of the sleeve. In the shown embodiment, the support member 102 has been fully inserted into the sleeve, and the support member 102 is sized similarly to the sleeve to hold the support member 102 in place when inserted.

In the illustrated embodiment, an opening is positioned at an end of the sleeve to allow for the support member 102 to be inserted into the sleeve. This allows for different support members 102 to be inserted into the sleeve. This also allows for the support member 102 to be removed so that the sleeve can be cleaned. In the shown embodiment, the opening can be sealed shut using a hook and loop fastener 201. This fastener will allow for the opening to be closed while still allowing the cushion to sit comfortably on a user's neck.

Referring now to FIG. 3, there is shown a perspective view of an embodiment of the article during use. The article is wrapped completely around a user's neck to provide support from all directions and to keep the head in a comfortable position. In this embodiment, the support member sits even with the jaw line of the user to support the head and neck area. In some embodiments, the support member can be made in various sizes in order to allow a user to customize a size of the support member to fit them best.

For example, the support member can be made with various lengths measuring between 15 and 35 centimeters. This range will ensure a size to fit children and adults, as well as males and females. In some embodiments, the support member may not need to completely encircle a user's neck. The support member for example may extend from a user's chin along the right jaw toward or to a position just before the spine. In such an embodiment, the user could lean slightly to the right side and have full support o prevent their head from tilting over in an uncomfortable position.

The support member can have any of a range of widths, so as to accommodate users with various neck lengths. The width can range anywhere from 9 to 20 centimeters. Other widths may be used for accommodation, depending on the size of the person, and 9 to 20 centimeters should not necessarily be interpreted as limiting.

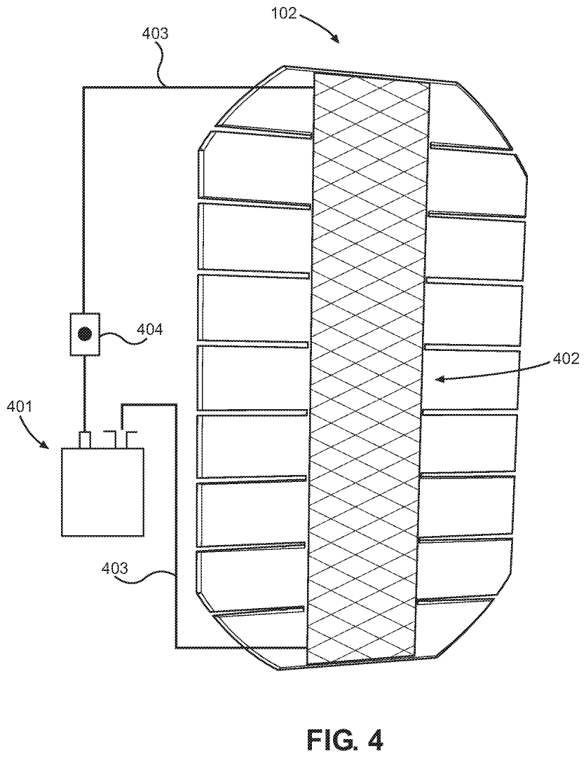

Referring now to FIG. 4, there is shown a perspective view of an embodiment of the support member with a heating element 402 attached. The heating element 402 can be warming fabrics. In this embodiment the warming fabrics can be glued onto the neck support. The fabrics are connected to a power source 401 using wires 403, and may be activated for use, for example, by actuating a push button switch 404.

By using such warming fabrics, the support member can remain thin and flexible while providing warmth to the user. Warming fabrics use electricity moving through them to create warmth, thus the old methods of heating such as coils and resistors are no longer needed. These fabrics remove the use of the large coils or wires as heating elements. In other embodiments a heating film can be used. This film is still thin and flexible.

In another embodiment the warming element can be a semi-conductive material. This material can provide heat through resistance to an electrical current. The semi-conductive material can be use as a coating. A layer can be added on the top of the support and attached to an electrical current providing heat similar to the heating fabrics. In another embodiment the semi-conductive material can be used to make up all or part of the support. The material can then be coupled to an electric current providing heat to the user.

In some embodiments, a power source 401 is required by warming fabrics to produce heat. In some embodiments, the power source 401 may be a small thin battery contained within the sleeve with the support. This eliminates the need for an external cord or an external power source. However, in other embodiments, the power source 401 can be external and attached via a cord. In such embodiments, larger batteries may be used, for example, to provide a greater battery charge.

Further, in some embodiments, an activation device is required. In one embodiment, a push button switch 404 can be used. This allow the push button switch 404 to be positioned on the sleeve and may contain the wires 403 inside of the sleeve. In other embodiments, a switch or button may be positioned outside the sleeve, for example, on or near an external power source. In some embodiments, an adjuster switch or knob may be used to control how warm the heating element is during use. In such an embodiment, the user may adjust the temperature according to preference or need.

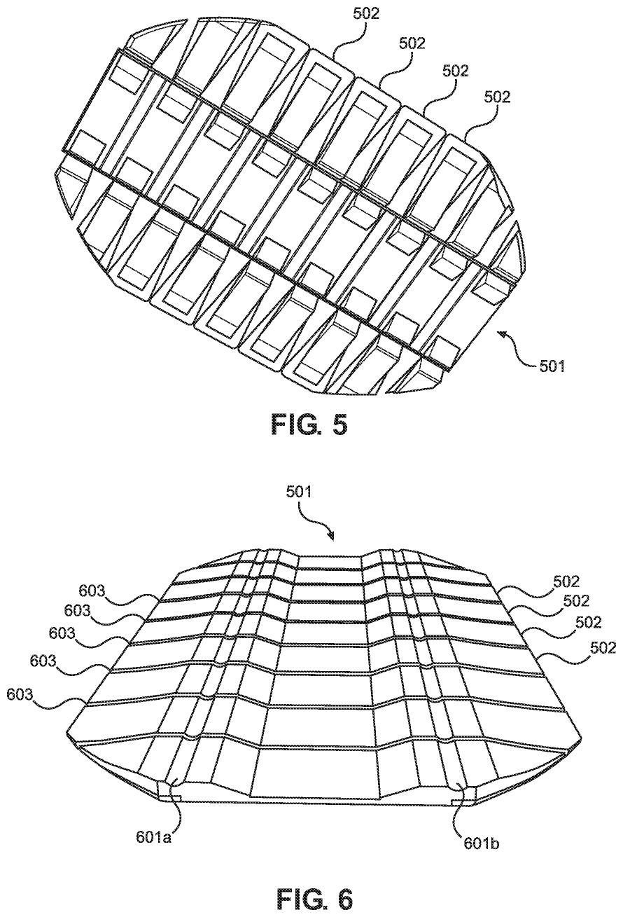

FIGS. 5, 6, 7, and 8 show several embodiments of various support members for the neck cushion and support article. The support members may be comprised of a spine 501 that is flexible. The spine may include a plurality of ribs 502 that are attached to the spine, which bend and shape to conform to the user's neck. In various embodiments, the ribs may be shorter further form a center portion of the support. This feature may form a curved shape or a diagonal line. In such embodiments, the support member may better conform to a user's cheek, chin, and jaw line to provide additional comfort and support to the user.

In some embodiments, the support member may be made out of various types of plastics. In one embodiment, the support member may be made out of Thermoplastic Polyurethane, also known as TPU. TPU has many desirable properties. It is a relatively low-cost material that may keep production costs down. TPU also has the ability to be used in a 3D printing process, which can be cheaper and faster than a molding process. This can ensure that the support member is made of one continuous material instead of having different interconnected parts. Finally, TPU provides a particular balance of flexibility, strength, and durability that may be desired in such support members. While TPU is discussed other flexible materials will also work for the make up of the support.

Referring now to FIG. 5, there is shown an embodiment of the support member, wherein a plurality of ribs 502 of the support member taper along a length thereof as they move away from the spine 501. This rib configuration can reduce the overall material needed to create the support. This reduced material can also reduce the weight of the support member, making the article more comfortable for the user.

Referring now to FIG. 6, there is shown an embodiment of the support member, wherein the support member includes grooves for easier bending of the support member during use. In this view, a height of a spine 501 is thin, and recessed compared to the ribs 502, where they connect to the spine 501. The spine 501 gradually thickens toward the rib connection points. This recession can reduce materials needed and weight. The ribs 502 can also be thinner as they move toward the end opposite the connection points. This can enhance comfort and reduce overall material needed.

Further, in FIG. 6, various grooves in the support member are shown.

There are grooves 601a and 601b on sides of the spine 501 to allow the ribs 502 to flex or bend easier. These grooves allow the ribs 502 to flex easier without applying extra pressure to the user during an adjustment. Further, there are grooves 603 that run across the spine 501. These grooves 603 line up with the separations in the ribs 502 and allow the support member to bend around the user's neck and head without added pressure. These grooves 603 also help to ensure that the support member is not stretched or bent out of place.

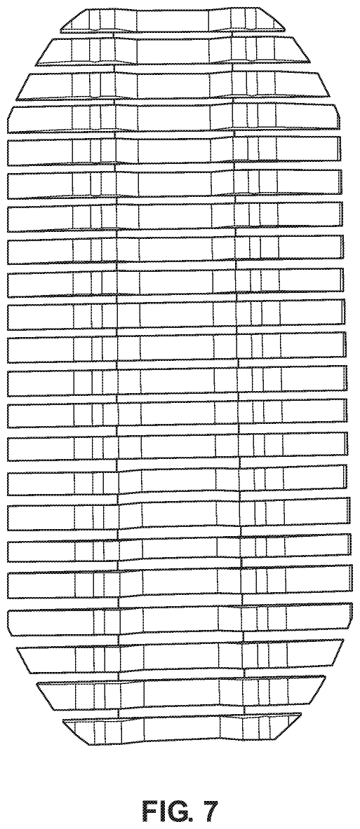

Referring now to FIG. 7, there is shown an embodiment of the support member, wherein the plurality of ribs is thinner, and wherein a width of the support member tapers toward the ends. In the shown embodiment, many more ribs are included compared to other embodiments. These ribs can be thinner as compared to other embodiments such that the overall length is not increased. The ribs can be of various thicknesses which will provide different advantages. With many thin ribs the article can add extra comfort to the user because the ribs can more closely conform to the user's neck. If the ribs are thicker this can provide added support. There can be any number of ribs having any of a plurality of different thicknesses, groove density, width and depth to determine the flexibility and supporting strength. In some embodiments, a combination of different thicknesses may be used.

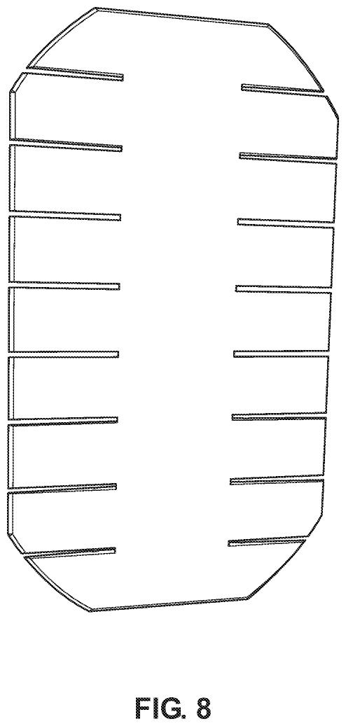

Referring now to FIG. 8, an underside of the exemplary embodiment of the support member is shown. The underside is depicted as completely flat with no added ridging. This will allow the support to be hidden from view and for the body to appear flat.

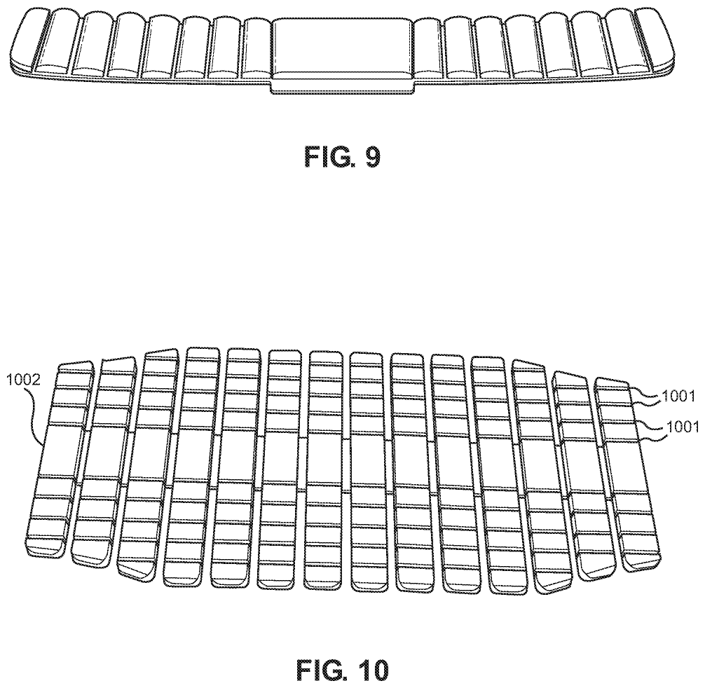

Referring now to FIG. 9, there is shown a perspective view of an embodiment of the support member with a cross section of a single rib. In this embodiment the combined single ribs support will be able to wrap around a user's neck and may not come up to the chin. In this embodiment the combining of single ribs, more comfort may be provided to user's who do not want the support member to be up and around the chin. In a further embodiment the support member may be thinker to provide more support. This can allow a user to have a stiffer support member. Referring now to FIG. 10, there is shown a front view of an embodiment of the support member with many grooves running through the ribs to provide flexibility. In one embodiment of the support member there can be a solid spine 1002 with a plurality of ribs placed thereon. The ribs may taper from the center to the edges of the spine 1002. In this embodiment there may be a plurality or grooves or indents 1001 running along the ribs. These grooves 1001 will allow the ribs to be more flexible. In this embodiment the ribs may bend more than in other embodiments.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1) An article for supporting an individual's neck, comprising:
a sleeve configured to receive a support member;
the support member comprises, a semi-ridged spine and a plurality ribs that are flexibly attached to the semi-ridged spine, wherein the semi-rigid spine includes a recess along the rib connections, configured to facilitate flexion of the plurality of ribs;
wherein the plurality of ribs is configured to support and conform to at least the individual's neck.

2) The article of claim 1, further comprising a heating element removably insertable into the sleeve, configured to warm at least the individual's neck during use.

3) The article of claim 1, further comprising an adjustable connector placed on the sleeve, wherein the adjustable connector enables a size of the article to be adjusted to secure the article to at least the individual's neck.

4) The article of claim 1, wherein a length of a selection of ribs of the plurality of ribs tapers along a length of the support member.

5) The article of claim 1, wherein the semi-rigid spine and the plurality of ribs are comprised of thermoplastic polyurethane.

6) The article of claim 1, wherein the semi-rigid spine and the plurality of ribs are unitary.

7) (canceled)

8) The article of claim 1, wherein the support member supports the individual's head, neck, and shoulder area.
